(19) United States
(12) Patent Application Publication
(10) Pub. No.: US 2008/0241926 A1
(43) Pub. Date: Oct. 2, 2008

(54) CELL ADHESION ON SURFACES OF VARYING TOPOGRAPHIES

(76) Inventors: Ilsoon Lee, Okemos, MI (US); Christina Chan, Okemos, MI (US); Srivatsan Kidambi, Cambridge, MA (US)

Correspondence Address: HARNESS, DICKEY & PIERCE, P.L.C., P.O. BOX 828, BLOOMFIELD HILLS, MI, 48303, US

(21) Appl. No.: 12/041,244

(22) Filed: Mar. 3, 2008

Related U.S. Application Data
(60) Provisional application No. 60/904,681, filed on Mar. 2, 2007.

(51) Int. Cl. C12N 5/06; B05D 3/00; B05D 1/36; B32B 5/00

(52) U.S. Cl. 435/395; 427/299; 427/402; 428/409

(57) ABSTRACT

Micro-topography of a surface influences cell adhesion and proliferation. To improve adhesion, polyelectrolyte multilayers (PEMs) are built on patterned support layers to increase surface wettability, thereby improving attachment and spreading of the cells. Physical parameters, such as pattern size and pitch, in part, regulate cell adhesion and proliferation. Varying the surface topography provides a method to influence cell attachment and proliferation for tissue engineering applications.

### CROSS-REFERENCE TO RELATED APPLICATIONS

This application claims the benefit of U.S. Provisional Application No. 60/904,681 filed on Mar. 3, 2007. The disclosure of the above application is incorporated herein by reference.

### U.S. GOVERNMENT RIGHTS

The work reported here was supported in part by NIH 1R01GM079688-01 and by NSF grants BES 0222747, BES 0331297, BES 0425821, and CTS 0609164. The United States Government may have some rights to this invention.

### INTRODUCTION

The present disclosure relates to cell adhesion on surfaces with varying topographies.

Cell-substratum interactions are important to many biological phenomena. Elucidating these interactions and how they may be controlled is crucial to understanding how to manipulate and design better biological systems and medical devices. Tissue engineering application is an example where control of these interactions is essential to the creation of functional engineered-tissues. (See for example, Langer et al., Science (Washington, DC) 260, 920-6 (1993); Patel et al., FASEB Journal 12, 1447-1454 (1998); and Boyan et al., Biomaterials 17, 137-46 (1996)).

The physical (such as surface irregularities, roughness) and chemical properties (such as hydrophilicity, charge) of a substrate affect the attachment and growth of cells. (See for example, Curtis et al., Journal of Biomaterials Science, Polymer Edition 9, 1313-1329 (1998); Tranquillo. R. T., Biochemical Society Symposium 65, 27-42 (1999); Evans et al., Journal of Biomedical Materials Research 40, 621-630 (1998); and van der Zijpp, Journal of Biomedical Materials Research, Part A 65A, 51-59 (2003)). Microtextured surfaces affect how the cells attach, spread and proliferate on these surfaces. (See for example, Dalby et al., Biomaterials 23, 2945-2954 (2002); and Wilkinson et al., Materials Science & Engineering C-Biomimetic and Supramolecular Systems 19, 263-269 (2002)). It is known that different surface chemistries of a material affect cell attachment. For example, Rubner and co-workers have examined the effect of surface chemistry on cell attachment and proliferation. (See for example, Yang et al., Biomacromolecules 4, 987-994 (2003); Yang et al., Abstracts of Papers of the American Chemical Society 224, U429-U429 (2002); and Yang et al., Abstracts of Papers of the American Chemical Society 226, U466-U466 (2003)). However, the role of surface topographical features on cell growth has been less well studied.

PDMS has been used extensively to study cell-substrate interactions, in medical implants and biomedical devices because of its biocompatibility, low toxicity, and high oxidative and thermal stability. PDMS is elastic, optically transparent, has low permeability to water, and low electrical conductivity. These properties, in addition to the ease with which it can be fabricated into microstructures using soft-lithography, have made this material attractive for use in cell biology studies, including contact guidance, chemotaxis, and mechanotaxis.

Despite the many advantages of PDMS, its applications in microfluidics and medicine have been problematic because PDMS is highly hydrophobic. Even when the surface is made hydrophilic, PDMS gradually reverts to its hydrophobic state due to surface rearrangements. As a result, it is rather difficult to maintain long-term culture of cells on PDMS, due to the difficulty in irreversibly modifying PDMS surfaces to have a stable cell-adhesive layer. Building a polyelectrolyte multilayer (PEM) film coating on top of the PDMS surface increases surface wettability and imparts lasting hydrophilicity thereby improving adhesion and proliferation of cells on PDMS surfaces. (See for example, Decher, G., Science 277, 1232-1237 (1997); Makamba et al., Analytical Chemistry 77, 3971-3978 (2005); and Ai et al. Cell Biochemistry and Biophysics 38, 103-114 (2003)). This method holds promise due to the ease with which these films can coat PDMS surfaces and the thickness of the films can easily be controlled. PEM is a simple method that allows formation of nanoscale structures by alternate adsorption of polyanions and polycations on virtually any substrate.

PEMs are excellent candidates for biomaterial applications due to (1) their biocompatibility and bioinertness, (2) their ease of incorporating biological molecules, such as proteins, and (3) their ease of control of the film structure and thickness, providing a simpler alternative for constructing complex 3D surfaces as compared with photolithography.

### SUMMARY

Various embodiments are based on an understanding of the cellular response to micropatterns (i.e., periodic microstructures), which is of significance to the design and application of biomaterials.

PEM-coated PDMS surfaces with different topographies affect the attachment, spreading and even proliferation of cells. Non-limiting examples of cells include mammalian cells such as transformed 3T3 fibroblasts (3T3s), HeLa (transformed epithelial) cells and primary hepatocytes. In an exemplary embodiment, the PEMs are built using LbL assembly of polyelectrolytes poly(diallyldimethylammoniumchloride) (PDAC), the polycation, and sulfonated poly(styrene) sodium salt (SPS), the polyanion, as shown in the scheme in FIG. 1A. Following cell seeding, differences in cell attachment and spreading depend on the nature of topographical features such as grooves and patterns on the PDMS surfaces. The cell morphology and attachment vary depending on the pattern geometries. Changes in the surface topographical features can be observed using imaging techniques and can alter the attachment and spreading of cells, suggesting a physical means of controlling the interaction between the cell and its environment.

In this way, cells adhere to a substrate in cytophilic regions and fail to adhere in cytophobic regions. The spatial arrangement of cytophilic and cytophobic regions is selected according to the requirements of the application at hand. In various embodiments, the substrates are made of an easy to make material such as silicone or polydimethylsiloxane (PDMS), which is turn coated with a PEM having an outer negative surface to provide a cytophilic surface, as described in Kidambi et al., J. Am. Chem. Soc. 2004, 126, 16286-16287, the full disclosure of which is incorporated by reference.

In an innovation, the surface is provided with a variation in surface topography. The topography is introduced in any suitable fashion. In a non-limiting embodiment, surface topography is applied on a silicon substrate by conventional photolithography. Then, the topography is transferred, in a negative sense, to a plastic substrate. In a preferred embodiment, PDMS components are poured onto the silicon substrate and then cured to form a removable plastic layer having topographical features that are the negative of those created in the silicon.

Surface features that are close to one another (i.e., where the pitch of the features is below the critical pitch) form a cytophobic region of the substrate, notwithstanding the substrate is covered with a nominally cytophilic coating. As the features of the topographical surface become closer to one another (i.e. as the “pitch” of the surface decreases), a point is reached at which the cells do not bind, forming a cytophobic region.

In various embodiments, cellular arrays on substrate are provided by exposing substrates of the invention to cells. The cells bind on the cytophilic regions and do not bind on the cytophobic regions. The cytophobic regions are made by providing the surface with appropriately high pitched topography, while the cytophilic regions are PEM covered and either contain no topographical features or contain topographical features farther apart than a critical interfeature distance. Such a distance can be determined for any particular cell type by routine experimentation in view of the current description of various embodiments of the invention.

### BRIEF DESCRIPTION OF THE DRAWINGS

FIG. 1 is (A) Schematic diagram illustrates the method for treating the PDMS surfaces with PEMs and culturing cells on the surfaces with different topographies. PEMs (PDAC/SPS)10 are built on top of the PDMS surface and cells are then seeded. (B) Illustration of the overlap of a cell and a flat area between the circle patterns. The diameter (d) changes while the center to center distance (a) remains constant for the features: a=18 μm. Region 1 represents the area between any six adjacent patterns (features). Region 2 represents a cell attached between the patterns. Region 3 is the cell nucleus.

FIG. 2 is a chemical structure of polyelectrolytes used to build the PEMs (A) PDAC (B) SPS.

FIG. 3 is phase contrast microscope images of circle patterns on PDMS surfaces of varying diameter (A) P1, diameter=1.25 μ, (B) P2, diameter=2.0 μm, (C) P3, diameter=3.0 μm, (D) P4, diameter=4.0 μm, (E) P5, diameter=5.0 μm, (F) P6, diameter=6.0 μm, (G) P7, diameter=7.0 μm, (H) P8, diameter=8.0 μm, (I) P9, diameter=9.0 μm. All the patterns have constant pitch distance (center to center) of 18 μm and height of 2.5 μm (Scale bar, 50 μm).

FIG. 4 is optical micrographs of HeLa, primary hepatocytes, and fibroblast after 3 days in culture on various surfaces. HeLa cells cultured on (A) PDMS (B) PEM coated smooth PDMS and (C) TCPS. Primary hepatocytes cultured on (D) PDMS (E) PEM coated smooth PDMS and (F) TCPS. Fibroblasts cultured on (G) PDMS (H) PEM coated smooth PDMS and (I) TCPS (Scale bar, 250 μm).

FIG. 5 is optical micrographs of primary rat hepatocytes after 3 days in culture on various surfaces: (A) TCPS (B) PEM coated smooth PDMS surfaces (C) P1, (D) P5 (E) P9 (Scale bar, 50 μm).

FIG. 6 is fluorescence micrographs of focal adhesion and actin cytoskeleton in fibroblasts revealed with triple labeling using TRITC-conjugated Phalloidin (staining F-actin), anti-Vinculin (focal contacts) and DAPI (nuclei) after 3 days in culture on various surfaces: (A) TCPS (B) PEM coated smooth PDMS surfaces (C) P1, (D) P5 (E) P9 (Scale bar, 50 μm).

FIG. 7 is fluorescence micrographs of focal adhesion and actin cytoskeleton in HeLa cells revealed with triple labeling using TRITC-conjugated Phalloidin (staining F-actin), anti-Vinculin (focal contacts) and DAPI (nuclei) after 3 days in culture on various surfaces: (A) TCPS (B) PEM coated smooth PDMS surfaces (C) P1, (D) P5 (E) P9 (Scale bar, 50 μm).

FIG. 8 is proliferation of cells on various surfaces at 8h, 24h and 72h after cell seeding (A) fibroblast (B) HeLa cells. Data represents mean±S.E. of three independent experiments (*p<0.05 compared with control TCPS surfaces).

FIG. 9 is a fibroblast proliferation on various surfaces (A) TCPS (B) PEM coated smooth PDMS surfaces (C) P1, (D) P5 (E) P9 (F) PDMS. Data represents mean±S.E. of three independent experiments.

FIG. 10 is HeLa proliferation on various surfaces (A) TCPS (B) PEM coated smooth PDMS surfaces (C) P1, (D) P5 (E) P9 (F) PDMS. Data represents mean±S.E. of three independent experiments.

### DETAILED DESCRIPTION

In one embodiment, the invention provides a substrate that contains at least one cytophilic and at least one cytophobic region. The substrate comprises a polyelectrolyte multilayer film on a support layer. In various embodiments, the cytophilic regions are characterized by the absence of topographical features or by the presence of topographical features with a minimum feature-to-feaure or interfeature distance greater than about 13 μm. In various embodiments, the polyelectrolyte multilayer comprises alternating layers of polycation and polyanion where a polyanion forms the surface of the substrate. The polyanion substrate of the polyanionic surface of the substrate is normally cytophilic. However, in various aspects of the invention, the substrate is provided with cytophobic regions despite the cytophilic nature of the polyanion by introducing topographical features into the substrate as discussed herein. In various further embodiments, substrates are provided that have cells adhered to cytophilic regions of the substrate.

In a related embodiment, a substrate of the invention has at least one cytophobic region and at least one cytophilic region and comprises a silicone support layer onto which is coated a polyelectrolyte multilayer. The silicone support is characterized by at least two patterns of topographical features, wherein a first pattern corresponds to the cytophobic region and a second pattern corresponds to the cytophilic region of the substrate. The presence of topographical features and the relative pattern, including the pitch and the interfeature distances determine whether the region associated with a pattern is cytophilic or cytophobic. The invention also provides for substrates having adhered cells as before.

In various embodiments, the invention provides methods of propagating cells by growing them as they are bound to cytophilic regions of substrates as described herein. Cells that can be propagated according to the methods include primary cells and transformed cells. Non-limiting examples of cells include HeLa, hepatocytes, and fibroblasts. In one embodiment, the substrate comprises a polyelectrolyte multilayer on a support layer; the cytophilic regions of the substrate are characterized either by no topographical features or by topographical features that have a feature to feature distance greater than an empirically observed minimum value, which in some embodiments is about 13 μm. In various embodiments herein, the support layer is conveniently made from a silicone such as polydimethylsiloxane.

Generally, methods of making the substrates involve introducing topographical features into a support layer and then applying a polyelectrolyte multilayer film on top of the support layer. A preferred support layer is made of silicone or polydimethylsiloxane (PDMS), because of its ease and versatility of manufacture.

In one embodiment, the substrates are fabricated by a method comprising applying a plurality of polyelectrolyte multilayers onto a support layer to make the substrate. The support layer before coating is characterized by a non-uniform distribution of topographical features, wherein the non-uniform distribution defines at least two patterns of topographical features in the support layer. Cytophilic regions of substrate correspond to polyelectrolyte multilayer coated regions of the substrate having a first pattern of these topographical features, while the cytophobic regions correspond to regions of the substrate having a second pattern of topographical features.

In various embodiments, the substrates are fabricated by providing a support layer having topographical features as described herein.

In another embodiment, the invention provides a method of modifying the cytophilic nature of the surface of at least one region of the substrate comprising an underlying solid layer coated with a cytophilic polyelectrolyte multilayer. The method comprises introducing topographical features into the underlying layer in the region to be modified, and coating the underlying layer that includes the topographical features with a polyelectrolyte multilayer. As described herein, topographical features in the region to be modified are characterized by an interfeature distance (also called feature-to-feature distance) that is sufficiently low to render the surface of the substrate cytophobic in that region. As discussed herein, in various embodiments, it has been found that such regions tend to be cytophobic if the interfeature distances are about 13 μm or less and tend to be cytoophilic if the interfeature distances are greater than 13 μm.

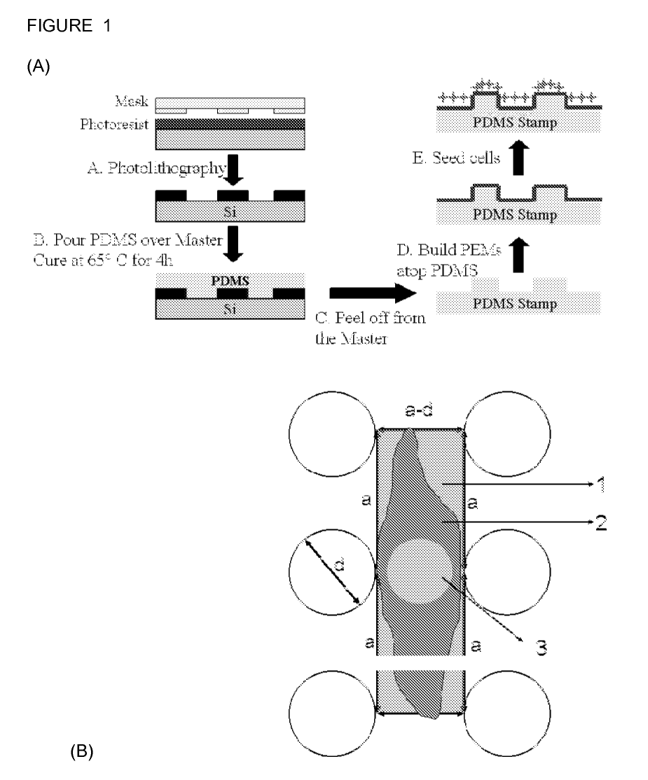

In various embodiments of the invention, the topographical features are arranged in a symmetric, uniform, or repeating pattern, at least in continuous regions defined in the surface of the substrate. When the topographical features are periodic, symmetric, or uniform, it is possible to characterize the topographical features by the area between six adjacent topographical features, as illustrated for example in FIG. 1B. In various embodiments, it has been found that if the area between six adjacent topographical features is about 500 μm² or less, the region tends to be cytophobic. In other embodiments, regions characterized by regular topographical features wherein the area between six adjacent topographical features is about 400 μm² or less are found to be cytophobic. In various preferred embodiments of the invention, the topographical features are cylindrical and have a height of about 0.5 to about 10 μm. In other embodiments, the topographical features have a height from about 1 to 5 μm.

The characteristic interfeature distance and interfeature area where the properties of the surfaces change between cytophobic and cytophilic can be determined experimentally or empirically for any cells or combination of cells that are being bound to the surface. Although the invention is not bound by theory, the values of distance and area appear to be related at least in part to the size of the cell that is binding.

In other embodiments, substrates containing cells adhered to the cytophilic regions as described herein are prepared by exposing such substrates to cultures of cells. As expected, the cells tend to bind or adhere to the substrate in cytophilic regions and do not bind at cytophobic regions. Binding of cells to polyanionic top layers of polyelectrolyte multilayer films is fairly general, and can include both primary and transformed cells. Non-limiting examples of cells include primary hepatocytes, HeLa cells, and fibroblasts.

The term cytophilic refers to surfaces on which cells tend to adhere, while cytophobic refers to surfaces which tend to resist cell binding. In one sense, the terms are relative in that if one surface binds better than another, it will be cytophilic relative to the other one, which is cytophobic. In another sense, the word cytophilic is used for surfaces that readily bind cells, while cytophobic is reserved for surfaces that essentially bind no cells. The result of binding cells to cytophilic or cytophobic regions of substrates described herein can be characterized by a cell density or a number of cells per unit area of the surface.

The binding of cells to an otherwise cytophilic surface has been found to depend on the existence or not of certain topographical features in the respective areas or regions of the substrate. Thus, when a region of a substrate has topographical features that have an interfeature distance of less than a certain amount, the surface tends to be cytophobic. On the other hand, when the substrate has either no topographical features or topographical features with an interfeature distance greater than a specified amount, the surface tends to be cytophilic. In one aspect then, the inherent cytophilicity of an outer layer of polyanion in a polyelectrolyte multilayer is altered by the polyanion being deposited on or coated on a support layer that has the topographical features. Topographical features that give rise to cytophobicity of the surface include those that amount to more than just normal surface roughness. In an illustrative embodiment, the topographical features are cylindrical in shape and are characterized by an average diameter of a cylinder and by an average height above the mean surface of the support layer.

The topographical features are also characterized by a distance between features and by a relative symmetry regularity or uniformness in the spacing of the topographical features on the one hand or random disposition on the other. In some preferred embodiments, the topographical features are “regular” in that they are evenly spaced from one another and form an extended two-dimensional pattern. In this aspect, a cytophilic region can contain a first pattern, regular or not, having typical interfeature distances greater than a specified amount. Similarly, cytophobic regions are characterized by a second pattern, regular or not, characterized by interfeature distances smaller than a specified amount. In various aspects, the interfeature distance at which topographical feature patterns transition from cytophilic to cytophobic depends on the nature of the cell being bound. It has been experimentally observed that the interfeature distance where this transition occurs is on the order of 13 μm for the cells tested.

When the topographical features of the support layer are regular, uniform, periodic, or repeating, it is possible to characterize the pattern of topographical features by the area between six adjacent typographical features. Such a spacing is illustrated for example in FIG. 1B. As can be appreciated, the area a between six adjacent topographical features depends on the interfeature distance a-d, which in turn depends upon the center-to-center distances of the topographical features and their extent or radius d.

Normally, the substrates described herein are fabricated by first incorporating the topographical features into a support layer, onto which a polyelectrolyte multilayer film is deposited by the known process of alternating deposition of polycation and polyanion. Cytophilic and cytophobic regions of the substrate thus made correspond to the topographical features present in the support layer. That is to say, the topographical features of the support layer are present in the same corresponding regions of the coated substrate.

A preferred support layer for substrates described herein is made of a silicone material such as polydimethylsiloxane or PDMS. Silicone support layers containing one or more regions of pattern topographical features can readily be made by photolithography on silicon, pouring a curable silicone solution over the silicon master, curing the silicone, and removing the silicone to provide a support layer having topographical features that are the negative of those etched into the silicon by photolithography. Photolithography can be readily used to provide topographical features spaced apart by distances on the order of μm. Such interfeature distances have been found useful for providing substrates having cytophilic and cytophobic regions as described herein.

Once the support layer containing the topographical features is provided, the polyelectrolyte multilayer films are deposited on the support layer by conventional means.

In a preferred embodiment, polyelectrolyte multilayer (PEM) films are deposited or coated on the support layer to provide a surface for cells to attach to. PEM films are formed by electrostatic interactions between oppositely charged poly-ion species. PEM are prepared layer-by-layer by sequentially immersing a substrate, such as a silicon, glass, or plastic slide, in positively and then negatively charged polyelectrolyte solutions in a cyclic procedure. Suitable substrates are rigid (e.g. silicon, glass) or flexible (e.g. plastics such as PET). A wide range of negatively charged and positively charged polymers is suitable for making the layered materials. Suitable polymers are water soluble and sufficiently charged (by virtue of the chemical structure and/or the pH state of the solutions) to form a stable electrostatic assembly of electrically charged polymers. Sulfonated polymers such as sulfonated polystyrene (SPS), anethole sulfonic acid (PAS) and poly(vinyl sulfonic) acid (PVS) are commonly used as the negatively charged polyelectrolyte. Quaternary nitrogen-containing polymers such as poly (diallyldimethylammonium chloride) (PDAC) are commonly used as the positively charged electrolyte.

Assembly of the PEM's is well known; an exemplary process is illustrated by Decher in Science vol. 277, page 1232 (1997) the disclosure of which is incorporated by reference. The method can be conveniently automated with robots and the like. A polycation is first applied to a substrate followed by a rinse step. Then the substrate is dipped into a negatively charged polyelectrolyte solution for deposition of the polyanion, followed again by a rinse step. Alternatively, a polyanion is applied first and the polycation is applied to the polyanion. The procedure is repeated as desired until a number of layers is built up. A bilayer consists of a layer of polycation and a layer of polyanion. Thus for example, 10 bilayers contain 20 layers, while 10.5 bilayers contain 21 layers. With an integer number of bilayers, the top surface of the PEM has the same charge as the substrate. With a half bi-layer (e.g. 10.5 illustrated) the top surface of the PEM is oppositely charged to the substrate. Thus, PEM's can be built having either a negative or a positive charge “on top”.

The current disclosure demonstrates that hydrophobic and cell resistant PDMS surfaces can be made to be cell adhesive surfaces by coating with PEM films. The addition of topographical features on the PEM coated surfaces provides an alternative approach to chemistry for controlling the attachment of primary cells (e.g. hepatocytes) and the attachment and growth of transformed cells (e.g. 3T3 fibroblasts and HeLa cells). The attachment and growth characteristics on the PEM coated PDMS surfaces are similar for the different cell types. In general, the rates of growth of the transformed cells on the PEM coated smooth PDMS surfaces without any topographical features are comparable to the growth on the control TCPS surfaces. The surface topographies, however, altered the attachment and spreading of the cell lines and primary hepatocytes as well as the proliferating ability of the cell lines.

PDMS is a useful material for cell biology studies because it can be easily manipulated into different sizes, shapes, and dimensions with soft-lithographic techniques. Differences in physical environment, e.g. the surface micro-topography on the PDMS surfaces, influenced the attachment and growth of the cells. Therefore, depending on the application requirements, the surface topography may be used as an alternative approach to chemical properties for controlling the attachment and growth of cells. These PDMS surfaces with varying topographies may be used, for example, to modulate fibroblast growth and spreading, which can be desirable in preventing conditions associated with fibroblast overproduction and overspreading. Overall, there are many advantages to fabricating devices made of PDMS, e.g., their low cost and ease of fabrication, and their biocompatibility and permeability to gas. Finally, as demonstrated in this study, PDMS when appropriately modified can be a suitable substrate for culturing and controlling the adhesion of various types of mammalian cells.

In various aspects, the present description provides for a method for patterning cells on a solid substrate. The present methods are useful for preparing surfaces that will enable investigators to screen and test for biologically active molecules that are capable of modulating the growth of cells through cell-surface interactions. As used herein, modulation can refer to adhesion of the cell, cell growth and cell differentiation, which can be examined using relatively small quantities of reagents and expensive growth factors and cytokines. Moreover, the screening of libraries of biologically active molecules, for example, peptide and gene libraries can be performed in high-throughput array format using the cell adhesive properties of the treated substrates described herein.

Cells have been shown to reproduce and form cellular networks on a variety of cell culture substrates. In some instances the growth rates of the cultured cells requires careful monitoring to ensure that the cell numbers do not exceed a certain level. The present substrates automate the growth of these cells by ensuring that the cells only adhere and attach to certain topographical patterns and not on others. One consequence is that cell adhesion and cell growth can be controlled automatically by altering the cytophilicity of the treated substrate, without regard to the chemical nature of the culture medium.

The invention has been described with reference various exemplary embodiments. Further non-limiting description is provided by the examples that follow.

### EXAMPLES

### 1. Materials

Poly(diallyldimethylammonium chloride) (PDAC) (Mw˜100,000-200,000) as a 20 wt % solution, sulfonated poly(styrene), sodium salt (SPS) (Mw˜70,000), fluorosilanes and sodium chloride were purchased from Aldrich (Milwaukee, Wis.). Poly(dimethylsiloxane) (PDMS) from the Sylgard 184 silicone elastomer kit (Dow Corning, Midland, Mich.) was used as substrates with varying topographies. The PDMS stamps were used for microcontact printing.⁴⁵ Dulbecco's Modified Eagle Medium (DMEM) with 4.5 g/l glucose, 10× DMEM, fetal bovine serum (FBS), penicillin and streptomycin were purchased from Life Technologies (Gaithersburg, Md.). Insulin and glucagon were purchased from Eli Lilly and Co. (Indianapolis, Ind.), epidermal growth factor from Sigma Chemical (St. Louis, Mo.). Adult female Sprague-Dawley rats were obtained from Charles River Laboratories (Boston, Mass.). Actin cytoskeleton and focal adhesion staining kit was purchased from Chemicon (Temecula, Calif.).

### 2. Preparation of PDMS Stamps

An elastomeric stamp was made by curing PDMS on a microfabricated silicon master, which acts as a mold, to allow the surface topology of the stamp to form a negative replica of the master (Decher et al. Thin Solid Films 244, 772-7 (1994). The PDMS stamps were made by pouring a 10:1 solution of elastomer and initiator over a prepared silicon master. (Kumar et al. Applied Physics Letters 63, 2002-4 (1993)). The silicon master was pretreated with fluorosilanes to facilitate the removal of the PDMS stamps from the silicon master. The mixture was allowed to cure overnight at 60° C. The masters were prepared in the Microsystems Technology Lab at MIT and consisted of circles with varying diameters with pitch distances of 18 μm and pattern heights of 2.5 μm as illustrated in FIG. 1B.

### 3. Preparation of Polyelectrolyte Multilayers

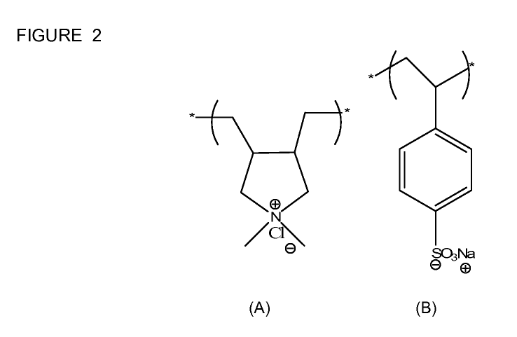

FIG. 2 shows the chemical structure of the polyelectrolytes namely SPS and PDAC used to build PEM films. PDAC and SPS polymer solutions were prepared with deionized (DI) water at concentrations of 0.02M and 0.01M, respectively, (based on the repeating unit molecular weight) with the addition of 0.1M NaCl salt. A Carl Zeiss slide stainer equipped with a custom-designed ultrasonic bath was connected to a computer to perform layer-by-layer assembly. Polyelectrolyte dipping solutions were prepared with DI water supplied by a Barnstead Nanopure-UV 4 stage purifier (Barnstead International Dubuque, Iowa), equipped with a UV source and final 0.2 μm filter. Solutions were filtered with a 0.45 μm Acrodisc syringe filter (Pall Corporation) to remove particulates. PDMS surfaces were subjected to a Harrick plasma cleaner (Harrick Scientific Corporation, Broading Ossining, N.Y.) for 3 min at 0.15 torr and 50 sccm flow of O₂ in a plasma chamber. To form the first bilayer, the PDMS substrates were immersed for 20 min in a polycation solution. Following two sets of 5 min rinses with agitation, the PDMS substrates were subsequently placed in a polyanion solution and allowed to deposit for 20 min. Afterwards, the PDMS surfaces were rinsed twice for 5 min each. The samples were cleaned for 3 min in an ultrasonic cleaning bath after depositing a layer of polycation/polyanion pair. The sonication step removed weakly bounded polyelectrolytes on the substrate, forming uniform bilayers. This process was repeated to build multiple layers. All experiments were performed using ten bilayers (i.e., 20 layers) with SPS as the topmost surface, thereby keeping the surface chemistry the same for all surfaces irrespective of the surface topography. Spectroscopic ellipsometry (model M-44, J. A. Woollam Co.) was performed according to a method previously described by Rubner and co-workers to obtain the thickness of the PEM film on top of the PDMS surface. (Nolte et al. Macromolecules 38, 5367-5370 (2005). The average thickness of a pair of PDAC/SPS film was determined to be approximately 3.7-4.0 nm. The PEM coated PDMS surfaces are dried before seeding the cells.

### 4. Cell Culture

4.1 Hepatocyte Isolation

Primary rat hepatocytes were isolated from 2 months old adult female Sprague-Dawley rats (Charles River Laboratories, Boston, Mass.), according to a two-step collagenase perfusion technique described by Seglen (Methods in Cell Biology 13, 29-83 (1976)) and modified by Dunn (Biotechnology Progress 7, 237-45.: (1991)). The liver isolations yielded 150-300×10⁶ hepatocytes. Using trypan blue exclusion the viability ranged from 90 to 98%. Primary hepatocyte culture medium consisted of DMEM supplemented with 10% FBS, 14 ng/ml glucagon, 20 ng/ml epidermal growth factor, 7.5 μg/ml hydrocortisone, 200 μg/ml streptomycin (10,000 μg/ml)—penicillin (10,000 U/ml) solution, and 0.5 U/ml insulin.

4.2 Hepatocyte Culture

The cells were seeded under sterile tissue culture hoods and maintained at 37° C. in a humidified air/CO₂ incubator (90/10 vol %). Primary hepatocytes were cultured on PEM coated 6-well TCPS. The multilayer coated TCPS plates were sterilized by spraying with 70% ethanol and exposing them to UV light before seeding the cells onto these surfaces. The cell culture experiments were performed on PEM surfaces without adhesive proteins. Collagen coated TCPS and uncoated TCPS were used as controls in these studies. A collagen gel solution was prepared by mixing 9 parts of the 1.2 mg/ml collagen suspension in 1 mM HCl with 1 part of concentrated (10×) DMEM at 4° C. The control wells were coated with 0.5 ml of this collagen gel solution and the coated plates were incubated at 37° C. for 1 hour. Freshly isolated hepatocytes were seeded at a concentration of 2×10⁵ cells per well and 2 ml were added to all the surfaces studied. One ml of fresh medium was supplied daily to the cultures after removal of the supernatant. Samples were kept in a temperature and humidity controlled incubator.

4.3 NIH 3T3, HeLa Cell Culture

NIH 3T3 fibroblast and HeLa cell lines were purchased from American Tissue Type Collection. Cells grown to 70% confluence were trypsinized in 0.01% trypsin (ICN Biomedicals) solution in PBS for 10 min and re-suspended in 25 mL media. Approximately 10% of the cells were seeded into a fresh tissue culture flask and the rest of the cells were used for the co-culture experiments. Fibroblast medium consisted of DMEM with high glucose, supplemented with 10% bovine calf serum and 200 U/mL penicillin and 200 μg/mL streptomycin. NIH3T3 and HeLa cells were seeded at a concentration of 2.5×10⁴ cells/ml and 2 ml were added to all the surfaces studied.

### 5. Cell Immunostaining

Cells were rinsed with PBS, followed by fixation with 4.0% paraformaldehyde in PBS for 20 min, rinsed three times in PBS, and then permeabilized with 0.1% Triton X-100 in PBS for 5 min and washed three times with PBS before adding the monoclonal antibody for vinculin. The cells were then washed three times with PBS and incubated with FITC-conjugated secondary antibody for vinculin and TRITC-conjugated phalloidin to label the actin filaments for 60 minutes. The cells were then washed three times with PBS and then incubated with DAPI for nuclei counterstaining for 5 minutes and washed again for three times with PBS. A Leica inverted phase contrast and fluorescence microscope with Soft RT 3.5 software was used to capture the images of the stained cells.

### 6. Determination of Cell Size and the Number of Cells on the Projected Area

The Soft RT 3.5 software was used on the phase contrast images of the cells to determine the average area occupied by a HeLa or mouse 3T3 cell. The surface area occupied by a typical cell on TCPS surfaces was measured from five different areas and repeated on three different substrates and then averaged for each surface. The number of cells on the projected cell area on the different surfaces was measured using the Image J software. The projected cell area refers to the area occupied by the cells as seen under the microscope. The number of cells per unit projected area was plotted over time for the various surfaces. The surfaces that supported proliferation showed a linear increase in the number of cells attached per unit area over time. The slope of this plot provided the rate of cell proliferation. To determine the amount of time for the cells to reach confluence, we first counted the number of fibroblasts or HeLa cells on the projected area at confluence. Then a theoretical amount of time was calculated for the cells to reach confluence by extrapolating the amount of time to reach the cell number at confluence from the above plot. This was confirmed visually under the microscope by observing when the cells reached confluence. Statistics was performed using the Student's t-test. A p value of 0.05 or lower was considered to be significant.

### Results and Discussions

### Fabrication of PDMS Substrate

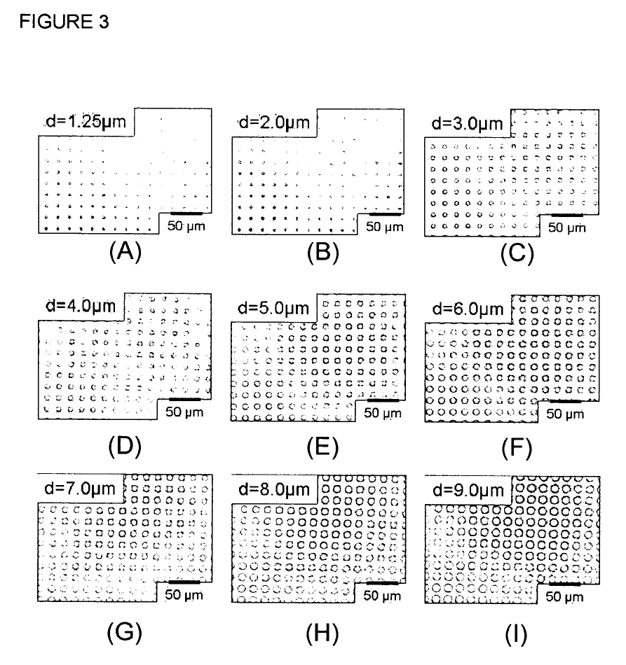

The dimensions and the topography of the patterns on the PDMS surfaces are shown in FIG. 1B, FIG. 3 and Table 1. The circle patterns have a pitch distance (center to center) of 18 μm while the diameter of the circle patterns ranges from 1.25 μm to 9 μm. The height of the patterns was 2.5 μm. The PDMS patterns were coated with PEMs (PDAC/SPS)₁₀ with SPS as the topmost surface. Thus, the variations in cell morphology and orientation on the different substrates were attributed to the surface topography rather than the surface chemistry.

Cell Attachment on PEM coated PDMS Surfaces

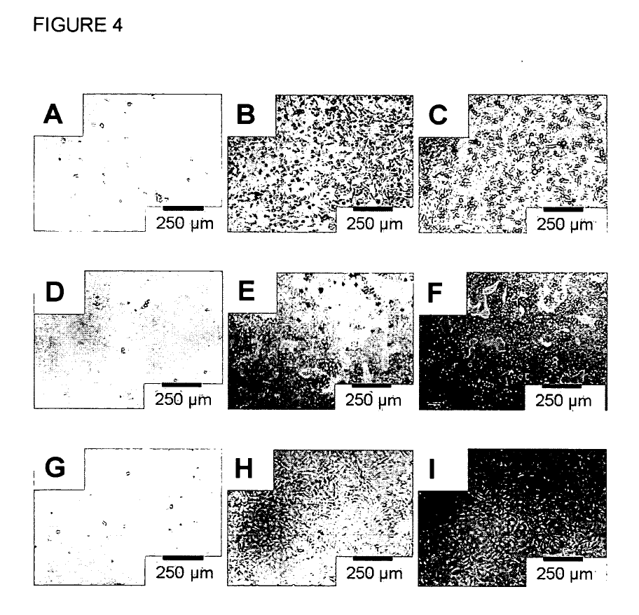

PDMS surfaces are highly hydrophobic and difficult to irreversibly modify these surfaces to have a stable cell-adhesive layer. As shown in FIGS. 4a, 4d, and 4g, the cells did not attach on PDMS surfaces. Coating the PDMS surfaces with (PDAC/SPS)₁₀ with SPS as the topmost layer improved the adhesion of primary hepatocytes, fibroblast and HeLa cells on the PDMS surfaces (FIGS. 4b, 4e, and 4h). To determine whether cells attached preferentially on a particular type of topography on the PEM coated PDMS surfaces, we evaluated the three different cell types on nine different surface topographies. The physical topographies varied in pitch distances (center to center of 18 μm) and pattern heights of 2.5 μm while the diameters of the circle patterns were 1.25 μm (P1), 2 μm (P2), 3 μm (P3), 4 μm (P4), 5 μm (P5), 6 μm (P6), 7 μm (P7), 8 μm (P8) and 9 μm (P9). All PDMS surfaces with varying topographies were coated with (PDAC/SPS)₁₀ with SPS as the topmost layer and adhesive proteins or ligands were not used. PEM coated PDMS surfaces without any topographical changes and tissue culture-treated polystyrene (TCPS) were used as controls. The cells were allowed to grow for up to 5 days on the different surface topographies. Each day, the cells on the different types of topographies were imaged using optical microscopy. At least five images were taken for each substrate, and at least three substrates were tested for each type of surface topography.

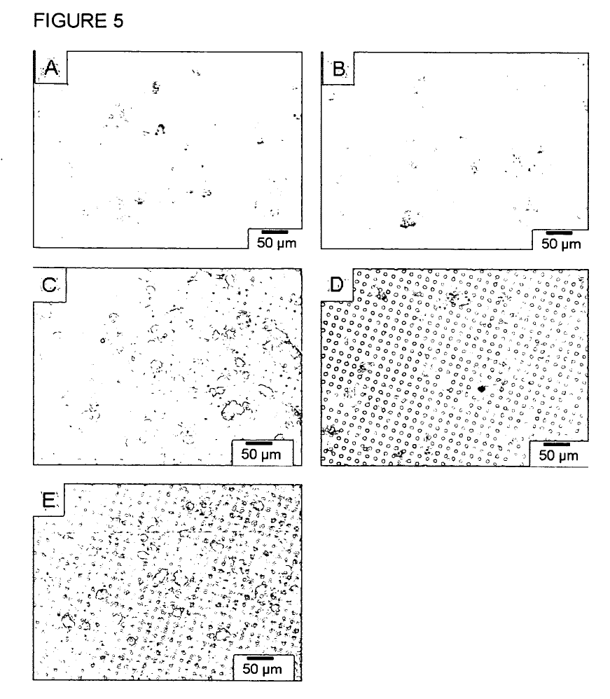

Primary Hepatocytes: The cells display different attachment preferences and morphologies depending on the pattern size and topography as shown in FIG. 5. The difference in the projected cell area for primary hepatocytes on the different topographies is shown in Table 2. There was a general trend of decreasing cell number with increasing diameter of the circular patterns and decreasing a-d distance. The number of primary hepatocyes that attached on the P1 (211-176 cells/mm²), P2 (201-164 cells/mm²), and P3 (191-155 cells/mm²) surfaces was comparable to the TCPS control (250-210 cells/mm²) and the PEM coated PDMS surfaces (245-200 cells/mm²), see FIG. 5 and Table 2. Cells on the P4-P9 surfaces showed more limited cell attachment, similar to the uncoated PDMS surfaces (98-5 cells/mm²), see Table 2. The cells on the (P4-P9) patterns did not spread and started to lift off over time resulting in a lower density of cells.

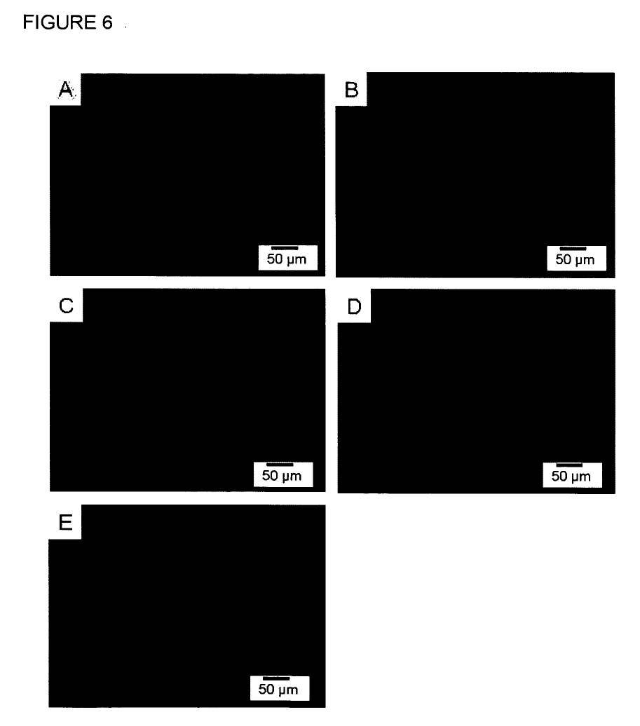

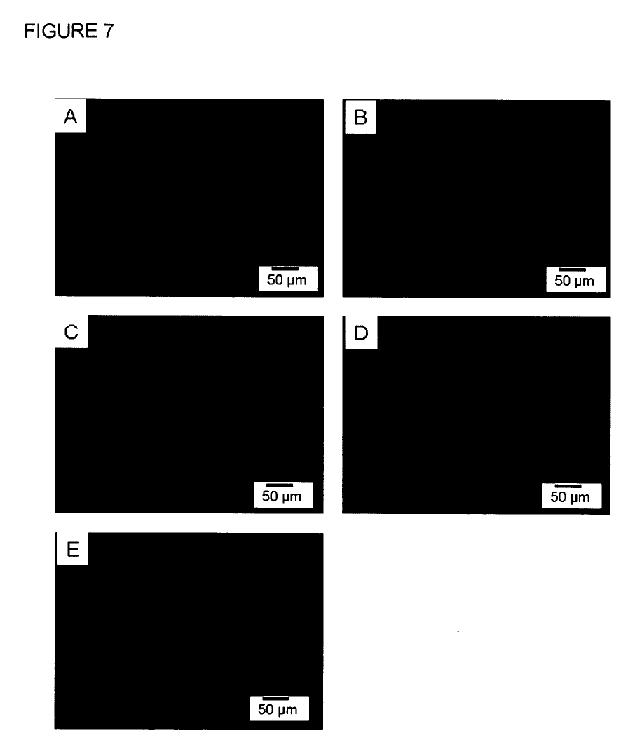

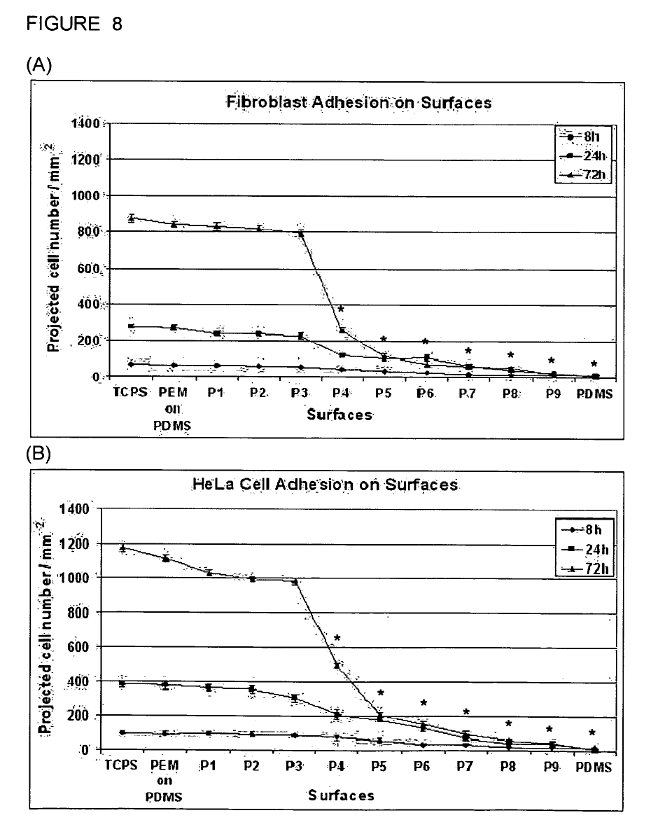

3T3 Fibroblast: The observations were similar when these micro-patterned PDMS surface topographies were cultured with fibroblasts. The cells display varying attachment preferences and morphologies depending on the pattern size and topography as shown in FIGS. 6 and 8. Fibroblasts showed varying cell adhesion depending on the diameter of the circle patterns and the a−d distance. On smooth PEM coated PDMS surfaces the morphologies and attachment patterns of the cells were similar to those on TCPS surfaces. On patterned PDMS surfaces, the cell attachment varied as the diameter of the circular patterns and the a-d distance changed. The cells attached preferentially on the smaller diameter patterns (P1, P2, P3), which had a correspondingly higher a-d distance, as compared to the patterns with the larger diameters and smaller a-d distance. On the P4-P9 surfaces where the cells detached, the cells appeared more rounded. The number of fibroblast cells that attached on the P1 (66-877 cells/mm²), P2 (62-865 cells/mm²), and P3 (57-841 cells/mm²) surfaces was comparable to the TCPS control (72-928 cells/mm²) and the PEM coated PDMS surfaces (69-893 cells/mm²), see FIG. 8 and Table 2. Hence, these PDMS topographies can be used to culture 3T3 fibroblasts, whereas PDMS surfaces P4-P9 were cytophobic to the fibroblast. Very few cells attached onto the uncoated PDMS surfaces (16-7 cells/mm²), see FIG. 8 and Table 2. HeLa Cells: The results for the HeLa cells were similar to the fibroblasts. A higher number of HeLa cells attached onto the P1 (98-1087 cells/mm²), P2 (93-1045 cells/mm²), and P3 (90-1036 cells/mm²) surfaces as compared to the P4-P9 surfaces, and was comparable to the TCPS control (101-1240 cells/mm²) and the PEM coated PDMS surfaces (95-1170 cells/mm²). Very few cells attached onto the uncoated PDMS surfaces (15-7 cells/mm²), see FIGS. 7 and 8 and Table 2. The coated PDMS surfaces were all covered with PEMs, with SPS as the topmost surface, thus the observed behavior is due to the surface topography.

Rate of Proliferation on PEM coated PDMS Surfaces

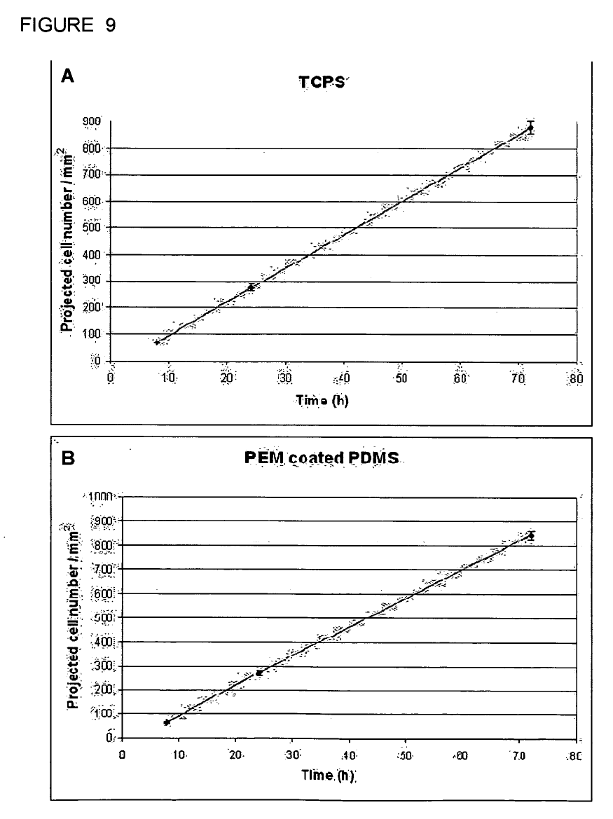

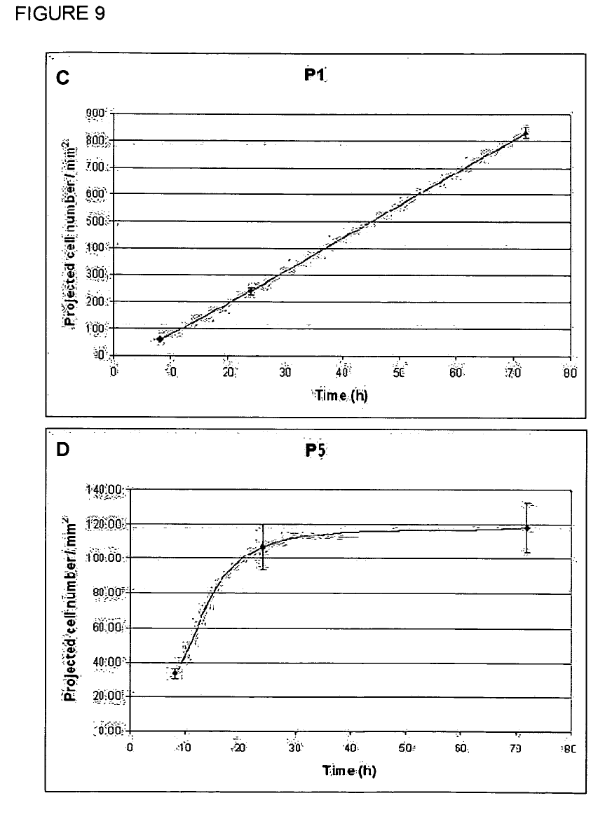

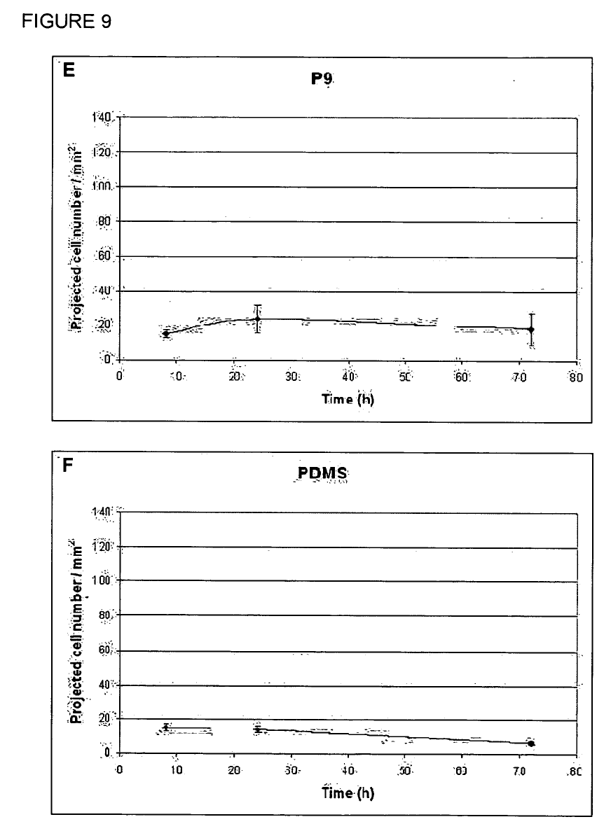

Fibroblast: The number of fibroblasts on the projected area was plotted against time as shown in FIG. 9. The surfaces which supported proliferation (TCPS, PEM coated PDMS, P1-P3) showed a linear increase in the number of cells that attached over time. This linear increase was observed for TCPS, PEM coated PDMS, P1, P2 and P3, but only TCPS, PEM coated PDMS and P1 are shown for illustration. The curves for P2 and P3 were similar to the curve for P1. FIG. 9 was used to determine the rate of proliferation on the various surfaces. Table 3 compared the rate of cell proliferation on the surface topographies that support proliferation (P1-P3 and P4) with those that do not (P5-P9) as well as on TCPS and PEM coated smooth PDMS. The rate of proliferation of the fibroblast on the P1-P3 surfaces (11-12 cells/mm²/h) were on par with the control TCPS and the PEM coated smooth PDMS surfaces (12-13 cells/mm²/h) as shown in Table 3. The rate of proliferation of fibroblasts on the P4 surface, although linear, was significantly slower than the control surfaces (4.6 cells/mm²/h) and did not reach confluence by day 5. The proliferation rate was much lower on the P5-P9 surfaces, and close to zero for the P9 surface (FIG. 9 and Table 3). Very few fibroblasts attached on the uncoated PDMS surfaces, thus the rate of proliferation was close to zero as illustrated in FIG. 9 and Table 3.

The number of fibroblasts on the projected area when they reached confluence was measured to be 1350 cells/mm². The theoretical amount of time for the fibroblast to reach confluence was calculated by extrapolating the amount of time to reach the cell number at confluence from FIG. 9. The amount of time for the fibroblasts to reach confluence was estimated to be between 4 to 5 days (see Table 3). It was confirmed visually under the microscope that the fibroblasts cultured on the P1-P3 samples grew to confluence by day 5, similar to the TCPS and the PEM coated smooth PDMS surfaces.

Fibroblast cells are present in almost all tissue types and organs and they play a central role in the support and repair of tissues and organs. When a tissue is injured or a device is implanted, the nearby fibroblasts proliferate and migrate into the affected area, and produce a large amount of collagenous matrix, which helps to isolate and repair the affected tissue.⁵¹ On the other hand, overgrowth and overspreading of fibroblasts can cause diseases such as liver cirrhosis and non-functional scar tissues.^{52, 53} Thus surfaces that can modulate fibroblast growth and spreading can be useful in preventing conditions, such as scar tissue formation associated with implanted medical devices or engineered tissue constructs.

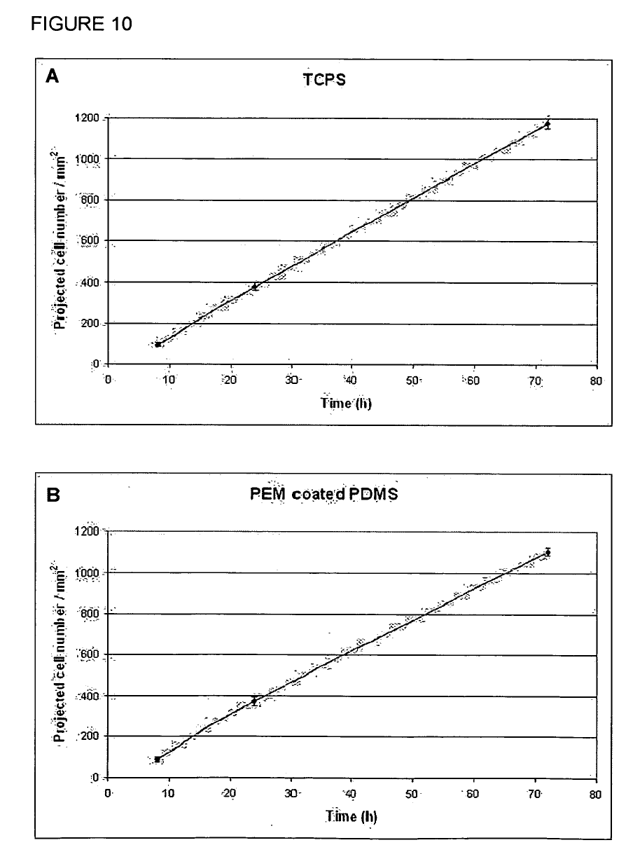

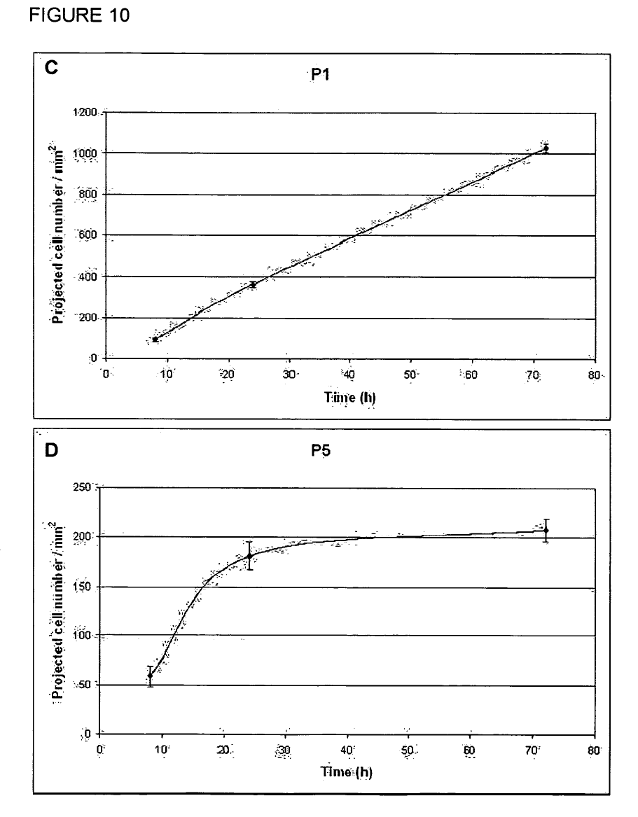

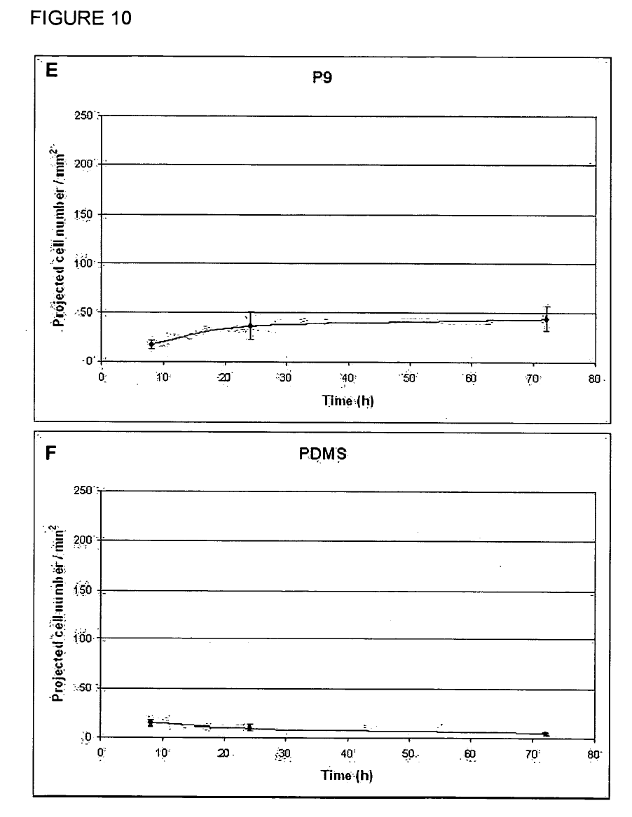

HeLa Cells: The number of HeLa cells on the projected area was plotted against time as shown in FIG. 10. The surfaces which supported proliferation (TCPS, PEM coated PDMS, P1-P3) showed a linear increase in the number of HeLa cells that attached over time, similar to that observed with fibroblasts. This linear increase was observed for TCPS, PEM coated PDMS, P1, P2 and P3. Although not shown, the curves for P2 and P3 were similar to the curve for P1. As with the fibroblasts, FIG. 10 was used to determine the rate of proliferation of the HeLa cells on the various surfaces. Table 3 compared the rate of cell proliferation on the various topographies. The rate of proliferation of HeLa cells on the P1-P3 surfaces (14-15 cells/mm²/h) were on par with the control TCPS and the PEM coated smooth PDMS surfaces (15-17 cells/mm²/h) as shown in Table 3. The rate of proliferation of the HeLa cells on P4 surfaces, although linear, was significantly slower than the control TCPS surfaces (6.4 cells/mm²/h) and did not reach confluence by day 5 despite a faster rate of proliferation than the fibroblast. The proliferation rate was much lower on the P5-P9 surfaces, and close to zero for the P9 surface (FIG. 9 and Table 3). Very few HeLa cells attached on the uncoated PDMS surfaces, thus the rate of proliferation was close to zero (FIG. 10 and Table 3).

The number of HeLa cells on the projected area when they reached confluence was measured to be 1650 cells/mm² on day 5. The theoretical amount of time for the HeLa cells to reach confluence was determined (as described above) to be between 4 to 5 days (see Table 3). This was confirmed visually under the microscope. The HeLa cells cultured on the P1-P3 samples grew to confluence by day 5, similar to the TCPS and the PEM coated smooth PDMS surfaces.

HeLa cells are virulent in nature, invade other cell cultures and result in the change of many continuous human cell lines into HeLa cell lines. These PDMS surfaces with varying topographies can be used to modulate HeLa cell growth and spreading, thereby potentially preventing them from invading other cell cultures.

### Potential Explanation of the Observed Effect of Topography on Cell Attachment

As seen from the data, the smaller diameter (1.25-3 μm) P1-P3 surfaces appeared to have higher cell proliferation rate when compared to the larger diameter (4-9 μm) P4-P9 surfaces. A possible explanation for the varying attachment and proliferation of the cells on the different topographies (P1-P9 surfaces) may be attributed to the difference in the area between the features. FIG. 1B is a schematic illustrating the overlap of a cell and a flat area between the circle patterns (features). Using the Soft RT 3.5 software on the phase contrast images of the cells we determined the average area occupied by a HeLa or mouse 3T3 cell (see Table 1). We observed that the area between the six adjacent circle patterns (features) decreased from 603±10 μm² for the P1 surface to 324±10 μm² for the P9 surface. The average area occupied by a HeLa or mouse 3T3 cell was measured to be 521±15 μm² (Region 2 in FIG. 1B).

The fibroblast and HeLa cells proliferated on the P1-P3 surfaces where the surface area between any six adjacent features (Region 1 in FIG. 1B) ranged from 603±10 μm² for the P1 surface to 540±8 μm² for the P3 surface. The P4 surface, on the other hand, has a surface area of 504±10 μm² which is on par with the size of an average cell. Even though the cells proliferated on the P4 surfaces, they proliferated significantly slower than on the control TCPS surfaces likely due to fewer cells being able to attach initially. The cells did not proliferate extensively on the P5-P9 surfaces where the surface area ranged from 468±μm² for the P5 surface to 324±10 μm² for the P9 surface, which are less than the average size of a cell. The cells proliferated on surfaces where the surface area between the features were larger than the size of an average cell (P1-P3), while the proliferation was slower on surfaces where the area between the features was on par with the size of the cells (P4) and did not proliferate extensively on surfaces where the area between the features was smaller than the average size of a cell (P5-P9). The results suggest the quantity of surface area between the features may affect the ability of the cells to attach, as well as the proliferation rate of the cells on the various topographies. Therefore, controlling the surface topography provides an alternative approach for modulating the cell attachment and proliferation for tissue engineering applications.

What is claimed is:

1. A method for modifying the cytophilic surface of at least one region of a substrate, the substrate comprising an underlying solid layer and a cytophilic polyelectrolyte multilayer, the method comprising:
introducing topographical features into the underlying layer in the region to be modified, and
coating the underlying layer including the topographical features with a polyelectrolyte multilayer,
wherein the topographical features in the region are characterized by an interfeature distance sufficiently low to render the surface of the substrate cytophobic in that region.

2. A method according to claim 1, wherein the interfeature distance is about 13 μm or less.

3. A method according to claim 1, wherein the topographical features are periodically spaced.

4. A method according to claim 3, wherein the surface area between six adjacent topographical features is about 500 μm² or less.

5. A method according to claim 3, wherein the surface area between six adjacent topographical features is about 400 μm or less.

6. A method according to claim 1, wherein the topographical features are cylindrical with a height of 0.5 μm to 10 μm.

7. A method according to claim 6, wherein the topographical features are cylindrical with a height of 1 μm to 5 μm.

8. A method according to claim 7, wherein the underlying solid layer is silicone.

9. A method of fabricating a substrate, the substrate comprising at least one cytophilic region and at least one cytophobic region, the method comprising
applying a plurality of polyeletrolyte multilayers onto a support layer to make the substrate,
wherein the support layer before coating is characterized by a non-uniform distribution of topographical features,
wherein the cytophilic regions of the substrate correspond to polyelectrolyte multilayer coated regions of the substrate having a first pattern of topographical features and the cytophobic regions of the substrate correspond to polyelectrolyte multilayer coated regions of the substrate having a second pattern of topographical features.

10. A method according to claim 9, wherein the first pattern is characterized by interfeature distances of 13 μm or less and the second pattern is characterized by interfeature distances of greater than 13 μm.

11. A method according to claim 9, wherein the first pattern comprises no topographical features and the second pattern is characterized by interfeature distances of less than 13 μm.

12. A method according to claim 9, wherein at least one of the first pattern and the second pattern is uniformly distributed.

13. A method according to claim 9, wherein the support layer is silicone.

14. A substrate comprising at least one cytophilic region and at least one cytophobic region, the substrate comprising a polyelectrolyte multilayer film on a support layer.

15. A substrate according to claim 14, wherein the hydrophilic region is characterized by no topographical features or by topographical features with a minimum interfeature distance of greater than 13 μm.

16. A substrate according to claim 14, wherein the cytophobic region is characterized by topographical features with a minimum interfeature distance of 13 μm or less.

17. A substrate according to claim 14, wherein the support layer is silicone.

18. A substrate according to claim 14, wherein the polyelectrolyte multilayer film comprises alternating layers of polycation and polyanion, and a polyanion forms the surface of the substrate.

19. A substrate according to claim 18, wherein the polyanion comprises sulfonated polystyrene.

20. A substrate according to claim 14, further comprising the cells adhered to the cytophilic region.

21. A substrate according to claim 20, wherein the cells are primary cells.

22. The substrate according to claim 20, wherein the cells are transformed cells.

23. A substrate according to claim 20, wherein the cells are selected from HeLa, hepatocytes, and fibroblasts.

24. A substrate having at least one cytophobic region and at least one cytophilic region comprising a polyelectrolyte multilayer film coated on a silicone support, wherein the silicone support is characterized by at least two patterns of topographical features wherein a first pattern corresponds to the cytophobic region and a second pattern corresponds to the cytophilic region.

25. A substrate according to claim 24, further comprising cells adhering to the cytophilic regions.

26. A substrate according to claim 25, wherein the cells are primary cells.

27. A substrate according to claim 25, wherein the cells are transformed cells.

28. A substrate according to claim 25, wherein the cells comprise HeLa, hepatocyte, or fibroblasts.

29. A method of propagating cells, comprising growing them as they are bound to a cytophilic region of a substrate, the substrate containing both cytophilic and cytophobic regions, wherein the substrate comprises a polyelectrolyte multilayer on a support layer, and the cytophilic region of the substrate is characterized either by no topographical features or by topographical features having a minimum interfeature distance greater than 13 μm, and the cytophobic region is characterized by topographical features having an interfeature distance of 13 μm or more.

30. A method according to claim 29, wherein the support layer is silicone.

31. A method according to claim 29, wherein the cells comprise primary cells.

32. A method according to claim 29, wherein the cells comprise transformed cells.

33. A method according to claim 29, wherein the cells comprise HeLa cells, hepatocytes, or fibroblasts.
